(19) United States
(12) Patent Application Publication
(10) Pub. No.: US 2021/0304856 A1
(43) Pub. Date: Sep. 30, 2021

(54) UNIVERSAL HOME TESTING SYSTEM FOR INFECTIOUS DISEASES

(71) Applicants: Agustin Ruiz Miller, West Hollywood, CA (US); Zen Charalambous, Miami, FL (US); Georgios Giorgallides, Las Vegas, NV (US)

(72) Inventors: Agustin Ruiz Miller, West Hollywood, CA (US); Zen Charalambous, Miami, FL (US); Georgios Giorgallides, Las Vegas, NV (US)

(21) Appl. No.: 17/216,451

(22) Filed: Mar. 29, 2021

Related U.S. Application Data
(60) Provisional application No. 63/049,496, filed on Jul. 8, 2020.
(60) Provisional application No. 63/005,021, filed on Apr. 3, 2020.
(60) Provisional application No. 63/002,204, filed on Mar. 30, 2020.

(51) Int. Cl. G16H 10/40; G06K 7/14; G06F 21/60

(52) U.S. Cl. CPC G16H 10/40; G06F 21/602; G06K 7/1417; G06K 7/1413

(57) ABSTRACT

A device and system used to enhance at-home medical test kits so that it provides the ability for a third-party system to capture test results, interacted contacts and other valuable information such as symptoms experienced. The goals are to provide the captured information to health authorities, medical partners and use it to execute contact tracing.

### CROSS-REFERENCE TO RELATED APPLICATIONS

This application claims benefit of priority under 35 U.S.C. § 119(e) of U.S. Provisional Patent Application Ser. No. 63/049,496, filed Jul. 8, 2020; U.S. Provisional Patent Application Ser. No. 63/005,021, filed Apr. 3, 2020; and U.S. Provisional Patent Application Ser. No. 63/002,204, filed Mar. 30, 2020, the contents of which are incorporated herein by reference in their entireties.

### BACKGROUND OF THE INVENTION

### Field of the Invention

The present invention relates generally to medical tests and more specifically to a system and method for performing diagnostic testing at home or near-patient testing, data management, including capturing the user's information, test results and location.

### Background Information

Infectious diseases are widely known to result in several hundreds of thousands of deaths each year in the United States alone. The emergence of endemics or pandemics is a constant threat to the viability of the economy and the national health system. When endemics or pandemics emerge, it is well documented that health systems suffer to respond both in terms of treatment as well as in terms of testing capacity and testing processing. It is further documented that testing is one of the first lines of defense, enabling key actions such as quarantine and contact tracing to stop the spread of the infectious disease.

Local, state and federal government often do not have the capacity to respond quickly to endemic or pandemic situations and as a result testing typically lags behind. Among the factors contributing to the lag include the prohibition of at home testing, the key assumption being that infectees will not always self-report out of fear to be stigmatized or limited in their movements; non-infectees on the other hand also present a reporting problem because once relief has been delivered, they find no reason to self-report. Due to this socio-cultural behavior, testing is limited to public and private labs or hospitals that are already taxed with treating patients. The result is severely delayed testing that has incredibly dangerous repercussions for local, state and federal economy.

Detection of analytes, particularly for drugs of abuse, is important in various settings, such as for certain workplaces and professions, as well as in law enforcement. Conventionally, collected samples are sent to a certified testing laboratory for analysis. However, sending the samples to the lab and then waiting for the lab to process, test the sample, and then report the results can take a significant amount of time, typically at least days. In many situations, it would be desirable to have testing results at the point of testing instead of waiting days for results from the lab.

Existing at-home tests show the results directly to the user, and the user can decide whether they want to self-report or not. At the same time, none of the existing at-home tests collect information necessary to do contact tracing.

Lateral flow assays require that the person reading the results have a certain degree of training to accurately interpret results, especially in scenarios with faint-color test lines that are barely detectable and easily confused with nitrocellulose pads. Relying on visual assessments presents significant problems since it is incredibly subjective varying from one person to another. Having a machine read that can objectively detect, process, and interpret results eliminates human error interpretation and increases the overall sensitivity and limit of detection of the assay.

Thus, there is a need for a way to enable the general population to rapidly and accurately self-test for infectious diseases, such as Covid-19, so they could quarantine themselves, and automatically self-report to the local authorities the fact that they took the test, whether they tested negative, or whether they tested positive, as well as provide a list of individuals they came in contact with so that the interacted individuals can be contacted and tested as well.

### SUMMARY OF THE INVENTION

The present invention is based on a detection device for reading, detecting, interpreting and encrypting test results, combined with a comprehensive data management system to improve diagnostics and for reporting and contact tracing.

Thus, in one embodiment, the invention provides a method and a detection device for reading, detecting, interpreting and encrypting the results of an at-home test kit or near-patient testing, displaying the results on an electronic system after the user has provided their personal information, and collecting the information needed to execute contact tracing. A new means for testing patients and capturing clinical data is urgently needed for the prevention of current spread and future outbreaks of infectious diseases, pathogens, drug exposure and the like.

Almost any substance or analyte can be detected by the system of the invention. In some aspects, infectious diseases such as those caused by bacteria, viruses, fungi, parasites and prokaryotic or eukaryotic organisms can be detected by the system and methods described herein, including but not limited to infectious diseases such as those caused by Ebola virus, Zika virus, influenza or coronaviruses such as Coronavirus Disease 2019 (COVID-19), SARS associated coronavirus (SARS-CoV), or Middle East respiratory syndrome coronavirus (MERS-CoV). However, it will understood that the invention is not limited to detection of infection diseases but rather encompasses analyte detection for medical diagnostics, industrial applications, veterinary applications, occupational testing and the like.

In another embodiment, the invention provides a detection device. The detection device includes: a) a testing module configured to perform an assay of a sample; b) a sensor for detecting a result of the assay; c) a control module operable to receive analyze and interpret test data from the sensor; and optionally d) an encryption module operable to encrypt test data; and optionally e) a communication module for transmitting test data to another computerized device. In certain aspects, the device further includes a user interface for interacting with the user and optionally displaying test results.

In yet another embodiment, the invention provides a detection device. The detection device includes: a) a testing module configured to perform an assay of a sample; b) an optical sensor operable to detect a result of the assay; c) a digital module operable to receive and convert data of the result from the optical sensor; d) a control module operable to receive converted data from the digital module; e) an image processor operable to receive data from the control module and analyze the result of the assay; and f) an encryption module operable to encrypt analyzed data. In certain aspects, the device further includes a user interface module operable to receive encrypted data and generate a unique identification marker for the encrypted data. In embodiments, the identification marker includes an embedded link which directs a computerized device of a user to a web address to collect information related to the assay results and information related to exposure of a subject to other humans.

In another embodiment, the invention provides a detection device including: a) a testing module configured to perform a lateral flow assay to detect an analyte; b) one or more optical sensors; c) a computer processor having functionality to analyze assay data; d) an encryption module operable to encrypt data; and optionally a user interface module operable to receive encrypted data.

In certain aspects, the detection device may include one or more of one of the following: scanner, camera, Bluetooth, near field communication (nfc), WiFi, usb wire, gsm chip, LTE, 3g, microphone, or the like. This allows communication between the device and another computerized device and/or a website or web address. In such aspects, a user need not use a third-party system such as a website or a computer application to see their test results. The results may be displayed on the test device. For example: 1) the user provides their personal information when they purchase the test device; 2) the user does the test, and the test device communicates via a communication module (e.g., 3G chip) to send the results to a third party system along with the unique identification marker; 3) the user's results are shown on the detection device; 4) if the results are positive, the user is notified via email, text, or the like to fill in the info needed for contact tracing.

In certain aspects, the test device is operable to collect information needed from the user on its own. As such, no third party system or website is necessary to fulfill this function. For example, in one aspect, the user enters their information through voice communication (e.g., into a microphone of the device) with the device.

In various aspects, the detection device is configured to be operably connected to another computerized device in any manner, including wirelessly or via a cable.

In yet another embodiment, the invention provides a method for detecting exposure of a subject to a pathogen or drug. The method includes: a) conducting an assay of a sample from the subject to detect an analyte indicative of exposure of the subject to a pathogen or drug using a detection device; b) encrypting data of the assay; c) generating a unique identification marker for the encrypted data; and d) transmitting the encrypted data to another computerized device.

In still another embodiment, the invention provides a method for detecting exposure of a subject to a pathogen or drug. The method includes: a) conducting an assay of a sample from the subject to detect an analyte indicative of exposure of the subject to a pathogen or drug using a detection device; b) encrypting data of the assay; c) generating a unique identification marker for the encrypted data including an embedded link to a web address; d) directing a computerized device to the web address via the embedded link; and e) transmitting the encrypted data to the web address. In certain aspects, the results of the assay are not transmitted to the subject before the results are transmitted to the web address and information from the subject related to exposure of the subject to other humans is obtained. In certain aspects, the method further includes notifying the individuals that were exposed to the subject when the assay result is indicative of exposure of the subject to a pathogen. Health authorities are also notified and provided the assay result and exposure information.

In yet another embodiment, invention provides a system. The system includes: a) a detection device configured conduct an assay of a sample from a subject to detect an analyte indicative of exposure of the subject to a pathogen or drug; and b) computer functionality operable to: encrypt data of the assay; generate a unique identification marker for the encrypted data; and transmit the encrypted data to a remote server.

### BRIEF DESCRIPTION OF THE DRAWINGS

FIG. 1 is flow chart setting forth an illustrative example of the methodology of the invention in one embodiment of the disclosure.

FIG. 2 is a perspective view of a detection device in an embodiment of the disclosure.

FIG. 3 is a perspective view of the detection device shown in FIG. 2.

FIG. 4 is a perspective view of the detection device shown in FIG. 2.

FIG. 5 is a perspective view of the detection device shown in FIG. 2.

FIG. 6 is a bottom view of the detection device shown in FIG. 2.

FIG. 7 is a side view of the detection device shown in FIG. 2.

FIG. 8 is an exploded top perspective view of the detection device shown in FIG. 2.

FIG. 9 is an exploded bottom perspective view of the detection device shown in FIG. 2.

### DETAILED DESCRIPTION OF THE INVENTION

The invention is based on the finding that the existing at-home medical test kits do not have the ability to capture the information of the user, report the results to the health authorities and perform contact tracing. These steps are vital such as in the case of a pandemic where the health authorities need the results of all tests, whether negative or positive and more importantly, know the contact information of whoever tested positive, as well as the people they came in contact with since they began to show symptoms.

Additionally, infectious diseases must be detected rapidly and frequently to contain viral spread. Therefore, detecting infectious diseases requires rapid, reliable and readily accessible detection methods. Since at-home users are not trained in the reading of test results, both speed and reliability are compromised. Therefore, there is a need for the test results to be parsed into machine-readable data within a device that can consistently interpret the test results accurately and rapidly. Machine readable data can further be enhanced through algorithmic interpretation of that data to achieve higher consistency and reliability than even trained lab technicians.

Existing at-home medical test kits display the results to the user immediately. Since the existing at-home medical test kits are not transmitting the results to a third-party electronic device, and since the user does not have the obligation to share the results with anyone else, the health authorities may never find out about a positive or negative test. If the authorities do not find about a positive test, contact tracing will not be performed, and the disease will continue to spread. If the authorities never find out about a negative test, the denominator can never be known and local, state and federal confirmed cases and case fatality rate will never be accurately known, all of which heavily impact policy.

There is a need for an at-home medical test kit that requires the user to always report their results with a trusted third-party electronic system. The third-party electronic system can report test results to the health authorities, and collect the information needed to then execute contact tracing. This third party-electronic system can be a custom device, a website or a mobile application, for example.

For such a test kit to work, the results will need to be hidden from the user until the user has given their contact information to the third-party electronic system. Existing at-home test kits do not have that functionality. Existing at-home test kits have the ability to execute the test and show the results, or execute the test, convert the results into an electronic form and then display the results on an electronic screen. This means the detection device and system of the present disclosure will not be used to create new testing methods or new ways to convert the test results into an electronic form, but the detection device and system will rather be used to enhance existing at-home test kits or new at-home test kits with the functionality we described herein. This statement assumes that the at-home test kit will have a way to convert the results to an electronic format, or otherwise the manufacturer of the at-home test kit will add it. This also assumes that for existing at-home test kits that the detection device of the present disclosure will be used, the manufacturer will make changes to hide the test results and add the detection device of the present disclosure inside the testing kit.

Accordingly, the invention provides a detection device which includes: a) a testing module configured to perform an assay of a sample; b) an optical sensor operable to detect a result of the assay; c) a digital module operable to receive and convert data of the result from the optical sensor; d) a control module operable to receive converted data from the digital module; e) an image processor operable to receive data from the control module and analyze the result of the assay; and f) an encryption module operable to encrypt analyzed data.

In some embodiments, the detection device for use with of the invention performs a lateral flow assay. As such, the disclosure provides a detection device including: a) a testing module configured to perform a lateral flow assay to detect an analyte; b) one or more optical sensors; c) a computer processor having functionality to analyze assay data; d) an encryption module operable to encrypt data; and optionally a user interface module operable to receive encrypted data.

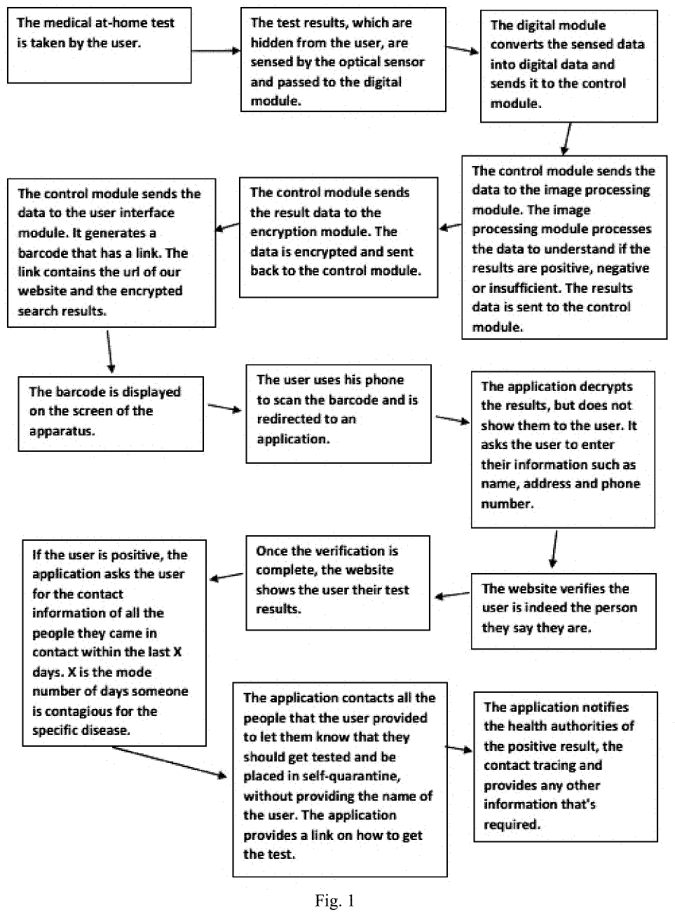

FIG. 1 is flow chart setting forth an illustrative example of the methodology of the invention in one embodiment of the disclosure. In some aspects, the method includes one or more of the steps set forth in FIG. 1. In some aspects, the method includes all of the steps set forth in FIG. 1.

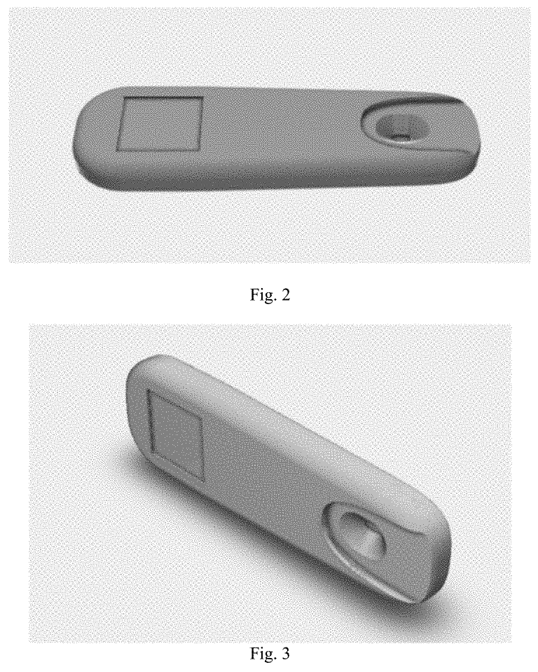

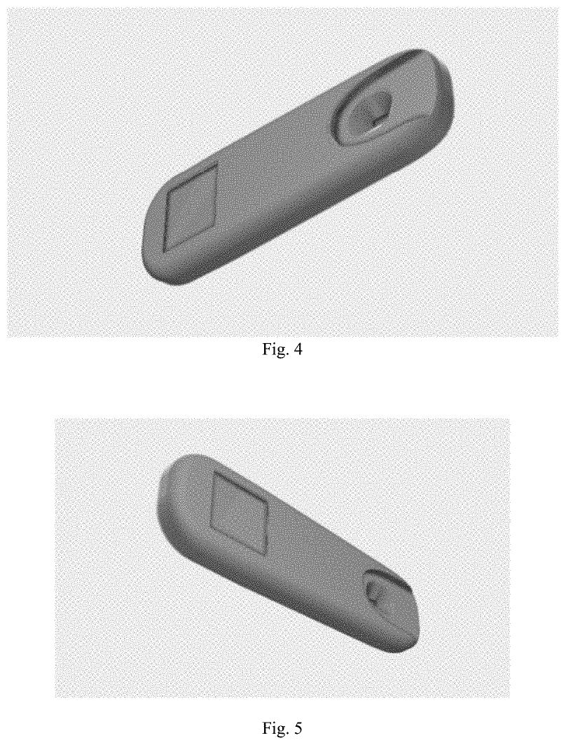

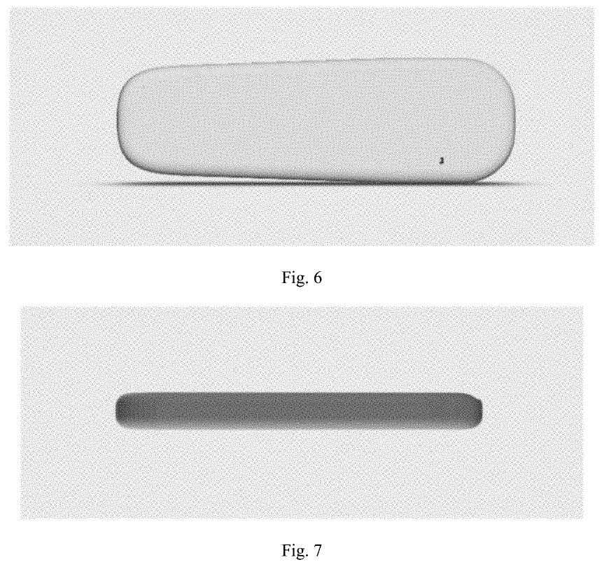

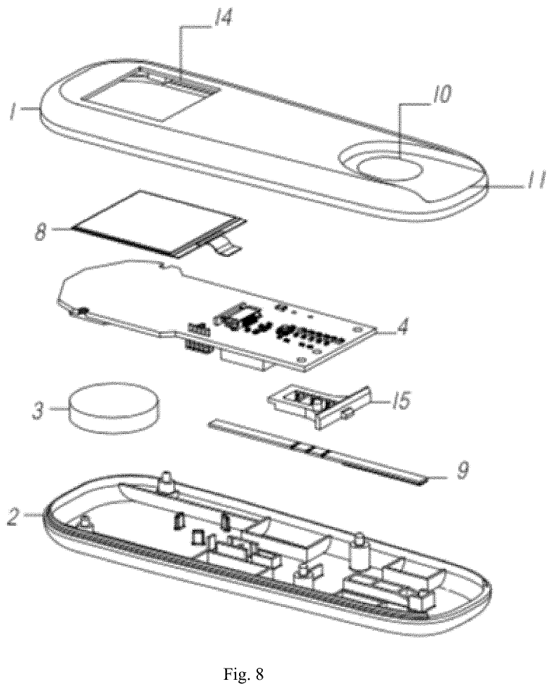

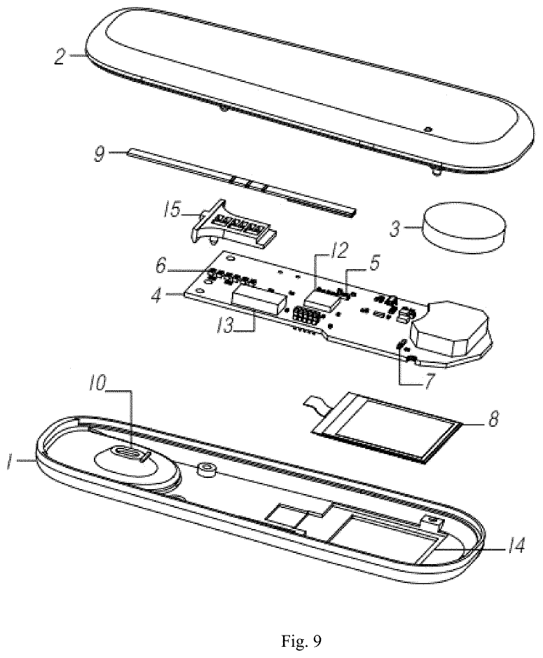

With reference to FIGS. 2-9, the detection device includes a two-part housing, formed of a synthetic polymeric material. The housing has a top part 1 and a bottom part 2. The housing is formed of an opaque plastics material such as polycarbonate or polypropylene. If necessary, an opacifier may be included. Within the housing is a power source, such as a small button cell battery 3, which delivers electrical power to the components mounted on a printed circuit board assembly (PCBA) 4 through a power supply, 5. These include, in particular, one or more optical sensors and LEDs 6, a phototransistor 7 and a liquid crystal display 8. The components mounted on the PCBA 4 include those necessary to read the results of the assay performed on the lateral flow immunoassay strip 9 mounted within the housing. The top and bottom parts 1, 2 of the housing co-operate to form a substantially moisture-impermeable seal around the aforementioned components. The phototransistor 7 activates the device at the presence of light and signals to the processor 12 to put the system in normal operation mode.

A blood sample reaches the lateral flow immunoassay strip 9 by means of a sampling well 10. The projecting portion of the sample well 11 is molded and dimensioned in the shape of a finger so as to cooperate with the user's efforts to direct the blood sample and buffer solution in the sample well. In the illustrated embodiment, to apply a blood sample to the sample well 10, the user places their finger on the molded portion 11 and the pricked part of their finger directly onto the sample well 10 to place the blood sample. Below the well, the immunoassay's sampling wick 9 is made of absorbent material and so the sample is absorbed into the wick. The user then deposits a buffer solution directly into the sample well 10 which is also absorbed into the wick and allows the blood sample to flow into the immunoassay strip 9.

In operation, the lateral flow assay proceeds in a conventional manner, resulting in the accumulation of a labelled binding reagent at a detection zone(s) on the test strip 9 which is detected and read by the assay reading components mounted on the PCBA 4. The optical sensors 6 increase the overall sensitivity of the assay, standardize results and eliminate interpretation errors. The processor 12 analyzes and processes the readings using an algorithm which is calibrated accordingly to reduce noise levels, detect to the lowest level of readability and overall enhance the reading of the lateral flow assay results. The results are then transferred into an encrypted package before displaying the barcode containing the encrypted results on the LCD 8 which is visible to the user via a window or aperture 14 formed at a suitable location in the top part 1 of the housing.

While the invention is illustrated using a lateral flow device as shown in FIGS. 2-9, it will be appreciated that the invention is not limited to this specific lateral flow device but rather may use a configuration of any known conventional lateral flow device that includes a sample receiving pad. Illustrative examples of lateral flow assay devices which may be utilized in the invention include, but are not limited to those disclosed in U.S. Pat. Nos. 10,073,091, 9,989,527, 9,709,562, 8,846,319, 9,944,922, 9,915,657, 8,822,151, 8,580,572, 8,153,444, 7,858,396, 7,910,381, 7,537,937, 7,344,893, 6,924,153, 6,372,513 and 6,656,744, each of which is incorporated herein by reference.

Additionally, while the disclosure illustrates use of a detection device configured to conduct a lateral flow assay, it will be appreciated that the testing module may utilize any number of detection modalities depending on the type of analyte to be detected. As used herein, an analyte may include, but is not limited to, a cell or portion thereof, cellular nuclei or portion thereof, a biomolecule, such as a nucleic acid (e.g., DNA, RNA, mRNA, tRNA or miRNA), amino acid, protein, peptide, hormone, steroid, lipid, carbohydrate or ion, a chemical compound, such as a small organic compound, a microorganism or portion thereof (e.g., virus, bacteria or fungi).

Whenever a user takes a test enhanced by the detection device of the present disclosure, the test results will be converted into an electronic format. This process is done by the optical module that's placed inside the test kit. The optical module consists of an optical sensor and LED array that collects the sensed data and sends it to the digital module where it's converted into digital data.

In some aspects, the digital module sends the data to the control module. The control module is responsible for passing the data between the four key modules: the digital module, the image processing module, the encryption module, and user interface module.

In some aspects, the control module sends the data to the image processing module. The image processing module applies a data processing algorithm that enhances the sharpness of the received data and translates the received data along a colorimetric index. The image processing module sends the processed results data back to the control module.

In some aspects, the control module sends the data to the encryption module. The encryption module encrypts the data and sends it back to the control module. The encryption module uses public key encryption. It has a public key that can be used to encrypt the data, and the system of the present disclosure has a private key that can be used to decrypt the data. This way if the detection device is compromised, and the public key is found, it still can't be used to decrypt the data.

In some aspects, the encrypted data is sent to a remote server with functionality to process and interpret data from the digital module to determine results of the testing. As such, the system need not include the image processing module and/or functionality to analyze data from the digital module.

In some aspects, the control module sends the data to the user interface module. The user interface module is responsible for displaying the data as a code, for example but not limited to a barcode or QR code. The code, e.g., the barcode will have a url to our system (e.g., website, app) and the url will contain the encrypted data. An example of a user interface module is a screen. The user interface module may optionally be a remote connection module such as bluetooth or NFC. One of skill in the art would recognize that while a barcode is an illustrative example, the barcode may be replaced with something else that has the same purpose: to help the user access the third-party system and easily transfer over the encrypted data.

In some aspects, the control module receives encrypted data from a remote server which is then decrypted by the control module. The user interface module can then be used to display the decrypted data.

In some aspect, the control module sends encrypted data to a remote which process and interpret data from the digital module to determine results of the testing. The data is then encrypted and sent back to the control module.

The user uses the information from the user interface module or the remote connection module to get access to the third-party system responsible for decrypting the test results. The third-party system decrypts the data using the private key, and guides the user through the collection of their personal information. To clarify, at the current state, the user is able to do so by scanning the barcode with his phone. The user's device internet browser or an app is launched. The user is able to see a message confirming that their test results are ready to be displayed (i.e. they have been decrypted), and the user can access them if they provide their contact information (e.g. name, address, phone).

Once the user enters their contact information, which could occur prior to or after scanning, the system uses up-to-date verification methods to make sure the user is who they say they are. Examples of different methods of verification vary from asking the user to take a picture of their ID or answering questions pertaining to their public information.

If the test is negative, the system stops here. If it's positive, the process continues.

The system asks the user for all the contact information of all the people they came in contact with in the last X days. The X is the mode number of days someone is contagious for the specific disease. Further, the system may also ask for other information that the authorities or medical partners deem valuable such as pre-existing medical conditions, incubation period, symptoms experienced, existing medications the subject is taking and the like.

The system contacts all the people that the user provided to let them know that they should get tested and be placed in self-quarantine, without providing the name of the user. The system provides a link on how to get the test.

The system notifies the health authorities of the positive result, the contact tracing and provides any other information that's required. The personal information of the user may be anonymized if the law allows for this and/or if the user makes such a request.

One of skill in the art would recognize that such test kits will include reagents and devices (e.g., cotton swabs, needle for a finger prick, tube for saliva, cup for urine) for obtaining a biological sample from the subject. Such samples may include blood, urine, saliva, nasal, or oral swabs, for example. In various aspects, a sample for use with the presently described invention is a biological sample which may be in liquid and/or solid form. In certain aspects, the sample is a biological fluid including but not limited to whole blood, serum, plasma, urine, feces bile, breast milk, breast fluid, gastric acid, mucus, pus, rheum, saliva, semen, sputum, sweat, tears, vaginal secretion, vomit, umbilical cord blood, tissue and endocervical fluid.

As discussed above, in addition to the infectious diseases already mentioned herein, it is understood that the system and methods of the invention can be used to detect any number of analytes, such as bacterial biological warfare agents. Such agents include, but are not limited to Bacillus anthracis (anthrax), Yersinia pestis (pneumonic plague), Franciscella tularensis (tularemia), Brucella suis, Brucella abortus, Brucella melitensis (undulant fever), Burkholderia mallei (glanders), Burkholderia pseudomalleii (melioidosis), Salmonella typhi (typhoid fever), Rickettsia typhii (epidemic typhus), Rickettsia prowasekii (endemic typhus) and Coxiella burnetii (Q fever), Rhodobacter capsulatus, Chlamydia pneumoniae, Escherichia coli, Shigella dysenteriae, Shigella flexneri, Bacillus cereus, Clostridium botulinum, Coxiella burnetti, Pseudomonas aeruginosa, Legionella pneumophila, and Vibrio cholerae.

Biological warfare fungus biowarfare agents include coccidioides immitis (Coccidioidomycosis). Biological warfare toxin genes capable of being detected by the methods of the present invention include botulism, T-2 mycotoxins, ricin, staph enterotoxin B, shigatoxin, abrin, aflatoxin, Clostridium perfringens epsilon toxin, conotoxins, diacetoxyscirpenol, tetrodotoxin and saxitoxin.

Biological warfare viral threat agents are mostly RNA viruses (positive-strand and negative-strand), with the exception of smallpox. These viruses mutate rapidly and the potential for engineered strains (natural or deliberate) is very high. RNA viruses cluster into families that have conserved RNA structural domains on the viral genome (e.g., virion components, accessory proteins) and conserved housekeeping genes that encode core viral proteins including, for single strand positive strand RNA viruses, RNA-dependent RNA polymerase, double stranded RNA helicase, chymotrypsin-like and papain-like proteases and methyltransferases.

Examples of (−)-strand RNA viruses include arenaviruses (e.g., sabia virus, lassa fever, Machupo, Argentine hemorrhagic fever, flexal virus), bunyaviruses (e.g., hantavirus, nairovirus, phlebovirus, hantaan virus, Congo-crimean hemorrhagic fever, rift valley fever), and mononegavirales (e.g., filovirus, paramyxovirus, ebola virus, Marburg, equine morbillivirus).

Examples of (+)-strand RNA viruses include picornaviruses (e.g., coxsackievirus, echovirus, human coxsackievirus A, human echovirus, human enterovirus, human poliovirus, hepatitis A virus, human parechovirus, human rhinovirus), astroviruses (e.g., human astrovirus), calciviruses (e.g., chiba virus, chitta virus, human calcivirus, norwalk virus), nidovirales (e.g., human coronavirus, human torovirus), flaviviruses (e.g., dengue virus 1-4, Japanese encephalitis virus, Kyanasur forest disease virus, Murray Valley encephalitis virus, Rocio virus, St. Louis encephalitis virus, West Nile virus, yellow fever virus, hepatitis C virus) and togaviruses (e.g., Chikugunya virus, Eastern equine encephalitis virus, Mayaro virus, O'nyong-nyong virus, Ross River virus, Venezuelan equine encephalitis virus, Rubella virus, hepatitis E virus).

Although the invention has been described with reference to the above examples, it will be understood that modifications and variations are encompassed within the spirit and scope of the invention. Accordingly, the invention is limited only by the following claims.

What is claimed is:

1. A detection device for detecting severe acute respiratory syndrome coronavirus 2 (SARS-CoV-2) comprising:
a) a testing module configured to perform an assay of a sample;
b) an optical sensor operable to detect a result of the assay;
c) a digital module operable to receive and convert data of the result from the optical sensor;
d) a control module operable to receive converted data from the digital module;
e) an encryption module operable to encrypt analyzed data; and optionally
f) an image processor operable to receive data from the control module and analyze the result of the assay.

2. The detection device of claim 1, further comprising a user interface module operable to receive encrypted data.

3. The detection device of claim 2, wherein the user interface module is operable to generate a unique identification marker for the encrypted data.

4. The detection device of claim 3, wherein the identification marker is a barcode or QR code.

5. The detection device of claim 3, wherein the identification marker includes an embedded link which directs a computerized device of a user to a web address.

6. The detection device of claim 5, wherein the encrypted data is transmitted to the web address.

7. The detection device of claim 1, wherein the testing module is configured to perform a lateral flow assay to detect SARS-CoV-2.

8. A method for detecting exposure of a subject to severe acute respiratory syndrome coronavirus 2 (SARS-CoV-2) comprising:
a) conducting an assay of a sample from the subject to detect an analyte indicative of exposure of the subject to SARS-CoV-2 using a detection device;
b) encrypting data of the assay;
c) generating a unique identification marker for the encrypted data; and
d) transmitting the encrypted data to a remote server.

9. The method of claim 8, further comprising processing the encrypted data on the remote server and transmitting encrypted data of the assay to the detection device.

10. The method of claim 9, wherein generating the unique identification marker for the encrypted data comprises generating an embedded link to a web address.

11. The method of claim 10, further comprising directing a computerized device to the web address via the embedded link.

12. The method of claim 11, wherein the encrypted data is transmitted to the web address which resides on the remote server.

13. The method of claim 8, wherein data of the assay is not transmitted to the subject.

14. The method of claim 10, further comprising collecting information from the subject through the web address.

15. The method of claim 8, further comprising verifying the identity of the subject.

16. The method of claim 15, further comprising transmitting data of the assay to the subject from the remote server.

17. The method of claim 16, further comprising collecting information from the subject related to exposure of the subject to other humans when the data of the assay indicated exposure of the subject to SARS-CoV-2.

18. The method of claim 17, further comprising notifying the other humans which were exposed to the subject and providing information regarding testing.

19. The method of claim 18, further comprising providing a medical or infectious disease authority with the information related to exposure.

20. A system comprising:
a) a detection device configured conduct an assay of a sample from a subject to detect an analyte indicative of exposure of the subject to SARS-CoV-2; and
b) computer functionality operable to:
encrypt data of the assay;
generate a unique identification marker for the encrypted data; and
transmit the encrypted data to a remote server.

21. The system of claim 20, wherein the computer functionality is further operable to process the encrypted data on the remote server and transmit encrypted data of the assay to the detection device.

22. The system of claim 21, wherein generating the unique identification marker for the encrypted data comprises generating an embedded link to a web address.

23. The system of claim 22, wherein the computer functionality is further operable to direct a computerized device to the web address via the embedded link.

24. The system of claim 23, wherein the encrypted data is transmitted to the web address which resides on the remote server.

25. The system of claim 20, wherein data of the assay is not transmitted to the subject.

26. The system of claim 22, wherein the computer functionality is further operable to collect information from the subject through the web address.

27. The system of claim 20, wherein the computer functionality is further operable to verify the identity of the subject.

28. The system of claim 27, wherein the computer functionality is further operable to transmit data of the assay to the subject from the remote server.

29. The system of claim 28, wherein the computer functionality is further operable to collect information from the subject related to exposure of the subject to other humans when the data of the assay indicated exposure of the subject to SARS-CoV-2.

30. The system of claim 29, wherein the computer functionality is further operable to notify the other humans which were exposed to the subject, provide information regarding testing to the user, and/or provide a medical or infectious disease authority with the information related to exposure.
